(19) United States
(12) Patent Application Publication
(10) Pub. No.: US 2023/0127168 A1
(43) Pub. Date: Apr. 27, 2023

(54) COMPOSITION, METHODS OF MAKING AND USING FOR TREATING A VIRAL INFECTION, INCLUDING CORONAVIRUS INFECTION

(71) Applicant: DEJIA HARMONY LLC, Leesburg, VA (US)

(72) Inventor: Zhonghua CI, Leesburg, VA (US)

(21) Appl. No.: 17/912,499

(22) PCT Filed: Mar. 17, 2021
(86) PCT No.: PCT/US2021/022672

§ 371 Date: Sep. 17, 2022

Related U.S. Application Data
(60) Provisional application No. 62/991,166, filed on Mar. 18, 2020.
(60) Provisional application No. 63/051,971, filed on Jul. 15, 2020.
(60) Provisional application No. 63/112,490, filed on Nov. 11, 2020.

(51) Int. Cl. A61K 36/355; A61K 36/634; A61K 36/258; A61K 36/538; A61K 36/808; A61K 36/736; A61K 36/483; A61K 36/484; A61K 47/44; A61K 9/00; A61K 9/16

(52) U.S. Cl. CPC A61K 36/355; A61K 36/634; A61K 36/258; A61K 36/538; A61K 36/808; A61K 36/736; A61K 36/483; A61K 36/484; A61K 47/44; A61K 9/0056; A61K 9/16

(57) ABSTRACT

A composition from Traditional Chinese Medicine (TCM) ingredients makes it possible to treat those infected with a virus, including, e.g., coronavirus or the flu, including but not limited to COVID-19 or Influenza A (“FluA”). Although makeable by other methods, the composition is makeable from a decoction of the TCM ingredients. Although usable for other purposes, the composition may be used to treat a patient in need of treatment for a virus, e.g., coronavirus infection, such as COVID-19 infection, or, e.g., the flu, such as FluA, by administering to the patient an effective amount of the composition.

### CROSS REFERENCE

The present application claims the benefits of priority to U.S. Provisional Application No. 63/112,490 filed 11 Nov. 2020, U.S. Provisional application No. 63/051,971 filed 15 Jul. 2020, and U.S. Provisional application No. 62/991,166 filed 18 Mar. 2020, each application of which is incorporated herein in its entirety.

### FIELD

A composition from Traditional Chinese Medicine (TCM) ingredients makes it possible to treat a patient infected with a virus, e.g., a coronavirus, including but not limited to COVID-19, or the flu, including but not limited to influenza A (“fluA”). Although makeable by other methods, the composition is makeable from a decoction of the TCM ingredients. Although usable for other purposes, the composition may be used to treat a patient in need of treatment for a viral infection, e.g., a coronavirus infection, such as, but not limited to, COVID-19 infection, or, e.g., the flu, including but not limited to fluA, by administering to the patient an effective amount of the composition. Although usable for other purposes, the composition may be used to clean surfaces.

### BACKGROUND

The new coronavirus disease (COVID-19) was a public health emergency of international concern. The effects of COVID-19 are apparent to anyone living though the emergency.

Moreover, coronavirus disease may be form SARS-CoV or SARS-CoV-2, whose effects are apparent to those in medicine.

In short, COVID-19 has swept through the international community with a ferocity not seen since the Spanish Flu in 1918. As of Mar. 15, 2020, there have been over 150,000 confirmed cases and 5,735 deaths worldwide. The World Health Organization has assessed the risk level as Very High both regionally and globally with the spread showing exponential growth and uncertainty prevailing over transmission mechanisms and effects of the virus.

COVID-19 is an acute respiratory illness with symptoms including fever, cough, and shortness of breath, with severe cases resulting in death. These symptoms are especially problematic for elderly individuals and those with pre-existing medical conditions including cardiovascular disease, chronic respiratory disease, and diabetes.

Humanity as a whole has struggled with how to deal with this extraordinarily contagious virus at the individual, community, country, and international levels. Thus far, there has been no way of stopping the spread of COVID-19. Governments have implemented isolationist policies limiting travel between countries and even imposed curfews to limit individual movement, shut down local businesses, and limit gatherings of crowds where the virus may be passed in large volumes. Hospitals have treated individuals for the symptoms as best they can with respirators, IV's, and other such attempts at minimizing the effects of the virus as it runs its course to prevent patients from dying, but no cure or effective treatment has been identified. Researchers are attempting to make a vaccine but there is as of yet no evidence of success or definitive results that contracting the virus multiple times is impossible, such that a vaccine may not work at all.

For over 2,000 years, Eastern Medicine has included the use of herbal remedies to improve human health status. Both in theory and in practice, specific herb combinations help dispel toxins from the human body and work as a preventative measure to build resilience and strengthen the body's immune system against viruses. As the CEO of the body, the lungs are one of the most important organs to fend off toxins and maintain health and functionality of all systems. Eastern medicine has focused on enabling the lungs to lead the body in these efforts, and herb combinations are especially effective when focused on supporting the lungs.

### SUMMARY

Objects and advantages may be made possible by the description which follows, and in part may be obvious from the description, or may be learned by practice thereof.

Accordingly, embodiments may provide novel methods and novel herbal combinations to combat the contracting and effects of influenza and COVID-19, along with related corona viruses and other viruses. Some embodiments may act to dispel toxins like influenza and COVID-19, along with related corona viruses and other viruses from the human body and works as a preventative measure to build resilience and strengthen the body's immune system against influenza and COVID-19, along with related corona viruses and other viruses. Additionally, the embodiments may also provide allergy and constipation relief, increase energy level and mental clarity, and improve sleep quality.

Nutraceutical and herbal formulations and the methods for preventing and/or mitigating influenza and COVID-19, along with related corona viruses and other viruses by the administration thereof. The nutraceutical compositions address the challenged state of the individual and potentiate their ability to prevent and mitigate the effects of contraction of influenza and COVID-19, along with related corona viruses and other viruses. The methods provide the formulation directly to the end organs where influenza and COVID-19, along with related corona viruses and other viruses affect the human body and its systems.

A method of treating a patient in need of treatment for coronavirus infection, comprising administering to the patient an effective amount of the composition disclosed herein. In some embodiments, the coronavirus infection is a SARS-CoV infection. In some embodiments, the coronavirus infection is from a SARS-CoV-2 infection. In some embodiments, the coronavirus infection is from COVID-19.

In some embodiments, the composition comprises a decoction of a precursor composition comprising Lonicera japonica; Forsythia suspensa; Panax ginseng; Schizonepeta tenuifolia; Scrophularia ningpoensis; Prunus armeniaca; honeycomb; Gleditsia sinensis; and Glycyrrhiza uralensis; or

a decoction of a precursor composition comprising the ingredients of composition #2 to 511 in table 1 (FIG. 8).

In some embodiments, the composition is in the form of a powder or paste made from the decoction whose supernatant aqueous phase is separated from the nonsoluble part of the precursor composition and is at least partially evaporated. In some embodiments, the supernatant aqueous phase is separated from the nonsoluble part of the precursor composition and is evaporated to dryness.

In some embodiments, the composition further comprises a pharmaceutically or dietary acceptable excipient. In some embodiments, the pharmaceutically or dietary acceptable excipient is chosen from solvents, binders, lubricants, herbal carriers, oils and salts.

In some embodiments, the composition is in a form suitable for oral administration. In some embodiments, the form suitable for oral administration is chosen from tablets, pellets, lozenges, granules, capsules, solutions, emulsions, and suspensions. In some embodiments, the form suitable for oral administration is a dietary supplement.

A precursor composition, comprises Lonicera japonica; Forsythia suspensa; Panax ginseng; Schizonepeta tenuifolia; Scrophularia ningpoensis; Prunus armeniaca; honeycomb; Gleditsia sinensis; and Glycyrrhiza uralensis; or

a precursor composition #2 to 511 in table 1 (FIG. 8).

In any embodiment, unless otherwise specified, the honeycomb is chosen from Polistes mandarinus Saussure, P. olivaceus, P. japonicus Saussure, and Parapolybia varia Fabricius.

A composition in the form of a powder made from the decoction whose supernatant aqueous phase is separated from the nonsoluble part of the precursor composition and is at least partially evaporated to form a powder or paste, in which the precursor composition, comprises Lonicera japonica; Forsythia suspensa; Panax ginseng; Schizonepeta tenuifolia; Scrophularia ningpoensis; Prunus armeniaca; honeycomb; Gleditsia sinensis; and Glycyrrhiza uralensis; or

a precursor composition #2 to 511 in table 1 (FIG. 8).

In some embodiments, the supernatant aqueous phase is separated from the nonsoluble part of the precursor composition and is evaporated to form a powder.

In some embodiments, the composition further comprises a pharmaceutically or dietary acceptable excipient. In some embodiments, the pharmaceutically or dietary acceptable excipient is chosen from solvents, binders, lubricants, herbal carriers, oils and salts. In some embodiments, the composition is in a form suitable for oral administration. In some embodiments, the form suitable for oral administration is chosen from tablets, pellets, lozenges, granules, capsules, solutions, emulsions, and suspensions.

Use of a composition described herein for treating a patient in need of treatment for a coronavirus infection.

Use of a composition described herein for making a medicament for treating a patient in need of treatment for a coronavirus infection.

A method of treating a patient in need of treatment for a viral infection, comprising administering to the patient an effective amount of the composition disclosed herein.

In some embodiments, the viral infection is a flu. In some embodiments, the flu is fluA.

In some embodiments, in any method, the composition comprises a decoction of a precursor composition comprising Lonicera japonica; Forsythia suspensa; Panax ginseng; Schizonepeta tenuifolia; Scrophularia ningpoensis; Prunus armeniaca; honeycomb; Gleditsia sinensis; and Glycyrrhiza uralensis; or

a decoction of a precursor composition comprising the ingredients of composition #2 to 511 in table 1 (FIG. 8).

In some embodiments, the composition is in the form of a powder or paste made from the decoction whose supernatant aqueous phase is separated from the nonsoluble part of the precursor composition and is at least partially evaporated. In some embodiments, the supernatant aqueous phase is separated from the nonsoluble part of the precursor composition and is evaporated to dryness.

In some embodiments, the composition further comprises a pharmaceutically or dietary acceptable excipient.

In some embodiments, the pharmaceutically or dietary acceptable excipient is chosen from solvents, binders, lubricants, herbal carriers, oils and salts.

In some embodiments, the composition is in a form suitable for oral administration. In some embodiments, the form suitable for oral administration is chosen from tablets, pellets, lozenges, granules, capsules, solutions, emulsions, and suspensions. In some embodiments, the form suitable for oral administration is a dietary supplement.

Use of a composition described herein for treating a patient in need of treatment for a viral infection.

Use of a composition described herein for making a medicament for treating a patient in need of treatment for a viral infection.

A method of disinfecting an animate object, comprise applying an effective amount of the composition to a topical surface of an animate object. In some embodiments, the composition is in the form of a gel, emulsion, lotion or cream. In some embodiments, the animate object is a human. In some embodiments, the surface is skin. In some embodiments, the skin is on a human hand.

Use of a composition described herein for disinfecting an animate object.

Use of a composition described herein for making a composition for disinfecting an animate object.

A method of disinfecting an inanimate object comprises applying an effective amount of the composition to a topical surface of an inanimate object. In some embodiments, the composition is in the form of a gel, emulsion, or a sprayable composition. In some embodiments, the inanimate object is chosen from tables, hard-backed chairs, doorknobs, light switches, phones, tablets, touch screens, remote controls, keyboards, handles, desks, toilets, and sinks. In some embodiments, the inanimate object is in a household setting. In some embodiments, the inanimate object is in an industrial or business setting. In some embodiments, the inanimate object is in a medical setting.

Use of a composition described herein for disinfecting an inanimate object.

Use of a composition described herein for making a composition for disinfecting an inanimate object.

Any use in which the composition comprises a decoction of a precursor composition comprising Lonicera japonica; Forsythia suspensa; Panax ginseng; Schizonepeta tenuifolia; Scrophularia ningpoensis; Prunus armeniaca; honeycomb; Gleditsia sinensis; and Glycyrrhiza uralensis; or

a decoction of a precursor composition comprising the ingredients of composition #2 to 511 in table 1 (FIG. 8).

A method, in which the composition comprises a decoction of a precursor composition comprising Lonicera japonica; Forsythia suspensa; Panax ginseng; Schizonepeta tenuifolia; Scrophularia ningpoensis; Prunus armeniaca; honeycomb; Gleditsia sinensis; and Glycyrrhiza uralensis; or

a decoction of a precursor composition comprising the ingredients of composition #2 to 511 in table 1 (FIG. 8).

A system as shown and described in the foregoing disclosure.

A method as shown and described in the foregoing disclosure.

An apparatus as shown and described in the foregoing disclosure.

A product as shown and described in the foregoing disclosure.

It is to be understood that the following detailed description is exemplary and explanatory only and are not restrictive of the invention, as claimed.

The accompanying drawings, which are incorporated in and constitute a part of this specification.

### BRIEF DESCRIPTION OF THE DRAWINGS

FIG. 1 illustrates formulations for the prevention and or mitigation of COVID-19 and the uses and methods of administration thereof.

FIG. 2 shows the results of VeroE6 cells, which were treated with serially diluted RDS, and infected with SARS-CoV-GFP pseudovirus.

FIG. 3 shows the results of A549 (ACE2) cells, which were treated with serially diluted RDS, and infected with SARS-CoV-GFP pseudovirus.

FIG. 4 shows a list of ingredients.

FIG. 5 shows the composition of example 1 (RDS) dosage-dependent inhibition of SARS-CoV-2(Luc) pseudovirus.

FIG. 6 shows the composition of example 1 (RDS) inhibited SARS-CoV-2(GFP) infection of A549(ACE2) cells. RDS inhibits SARS-CoV-2(GFP) pseudovirus entry into A549(ACE2) cells. A549(ACE2) cells were treated with serially diluted RDS, and infected with SARS-CoV-2(GFP) pseudovirus for 6 hours. Cells were washed to remove the virus and RDS, and cultured in the absence of RDS. Inhibition of viral infection was quantified at 72 hours post infection by flow cytometry. Uninfected Cell and SARS-CoV-2(GFP)-infected but RDS-untreated cells were used as controls. The percentages of GFP+ cells are shown. PI, propidium iodide.

FIG. 7 shows the composition of example 1 (RDS) inhibits FluA (GFP) virus infection of MDCK cells. MDCK cells were treated with serially diluted RDS, and infected with FluA (GFP) virus for 6 hours. Following infection, cells were cultured in the presence of RDS. Inhibition of viral infection was quantified at 36 hours post infection by flow cytometry. Uninfected Cell and FluA (GFP)-infected but RDS-untreated cells were used as controls. The percentages of GFP+ cells are shown. PI, propidium iodide.

FIG. 8 is a table showing 512 precursor composition options. In the table, 1-means the ingredient is present in the composition; and 0-means the ingredient is not present in the composition. The ingredients are as follows: Lonicera japonica (#1); Forsythia suspensa (#2); Panax ginseng (#3); Schizonepeta tenuifolia (#4); Scrophularia ningpoensis (#5); Prunus armeniaca (#6); honeycomb (#7); Gleditsia sinensis (#8); and Glycyrrhiza uralensis (#9).

### EMBODIMENTS

Reference will now be made in detail to embodiments, examples of test results for which are in the accompanying drawings.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiment(s) of the invention”, “embodiment(s)” or “invention” do not require that all embodiments of the invention include the discussed feature, advantage or mode of operation.

Some embodiments address the challenged functions of the body infected with various viruses and diseases, such as influenza and COVID-19, along with related corona viruses and other viruses, which are overwhelmed as the virus spreads from person to person and to different parts of the body, by providing the nutraceuticals that support the molecules. The embodiments may work to support the lungs, heart, immune system, and brain. The embodiments may also provide nutraceuticals elsewhere in the body to the end organs where the virus affects the body. By strengthening these core organs and systems within the body, the embodiments work to prevent the contraction or and mitigate the effects of influenza and COVID-19, along with related corona viruses and other viruses. The embodiments may adjust body functions, help restore balance in human body, and/or improve body immune functions by enabling itself to dispel pathogenic factors through perspiration, purgation and emetic therapy.

An embodiment includes a formulation of prevention and/or mitigation of influenza and COVID-19, along with related corona viruses and other viruses. The formulation may include herbs and other nutraceuticals. Exemplary herbs and nutraceuticals suitable for the composition include honeysuckle (1), Forsythia suspensa (2), suncured ginseng (3), schizonepeta (4), Scrophularia ningpoensis (5), apricot kernel (6), honeycomb (7), Gleditsia sinensis (8), and/or liquorice (9). The exemplary combination of herbs and nutraceuticals is based on supporting biological function and mechanism of interest in combatting contraction and symptoms of influenza and COVID-19, along with related corona viruses and other viruses, such as providing support to the lungs, brain, immune system, and heart.

Precursor Ingredients

A precursor composition comprises combinations of one or more ingredients selected from the following 9 TCM ingredients: Lonicera japonica (1); Forsythia suspensa (2); Panax ginseng (3); Schizonepeta tenuifolia (4); Scrophularia ningpoensis (5); Prunus armeniaca (6); Honeycomb (7); Gleditsia sinensis (8); and Glycyrrhiza uralensis (9).

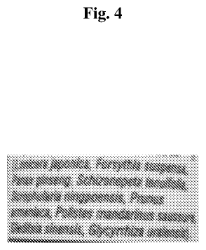

In some embodiments, the precursor composition comprises the listed ingredients in FIG. 4.

All these 9 ingredients and decoction thereof are available from vendors such as Shanghai Cai Tong De Pharmacy, China and others.

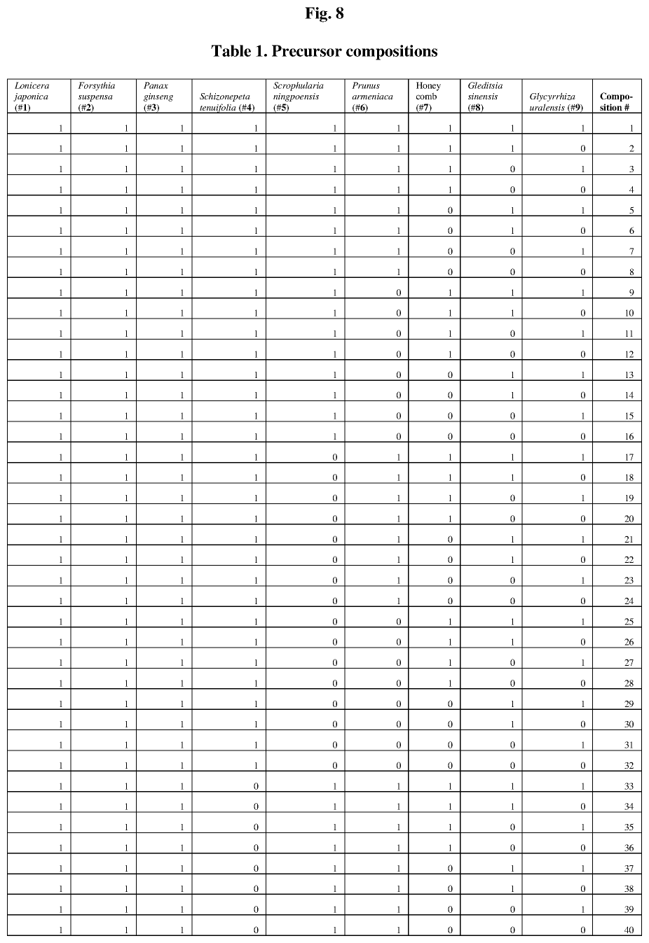

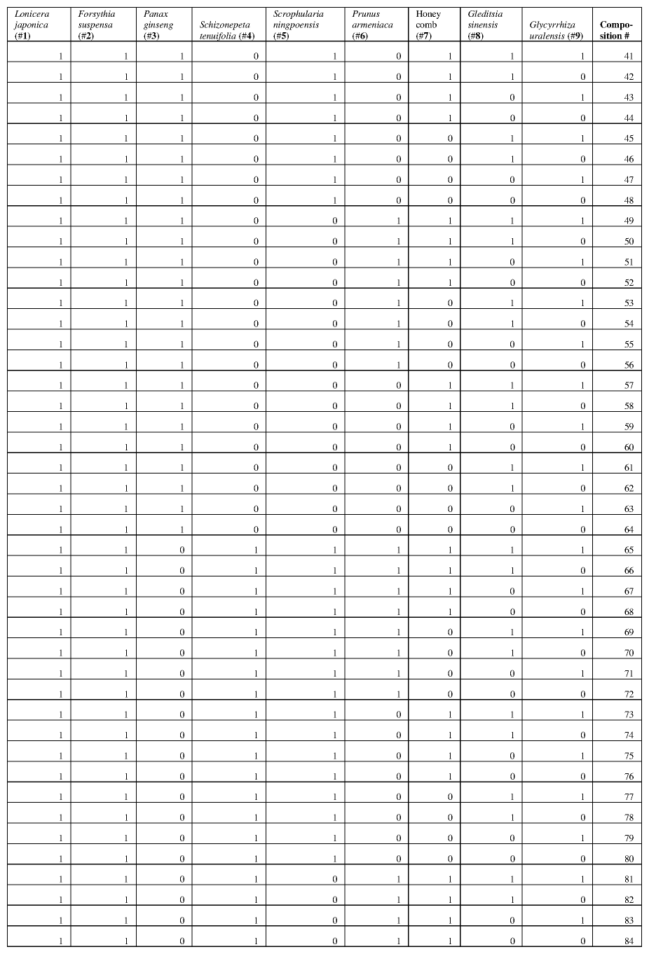

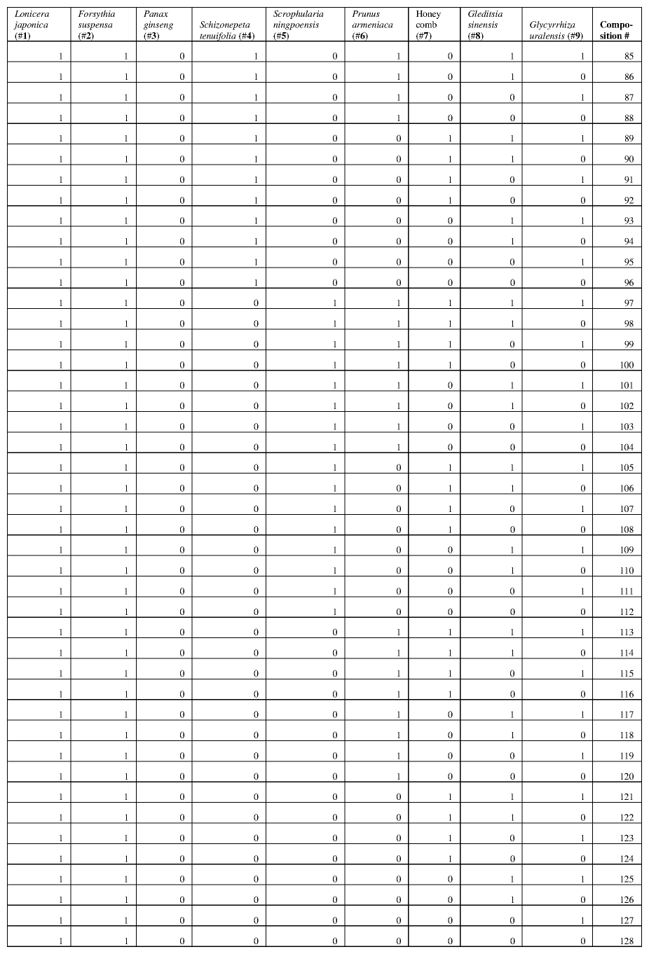

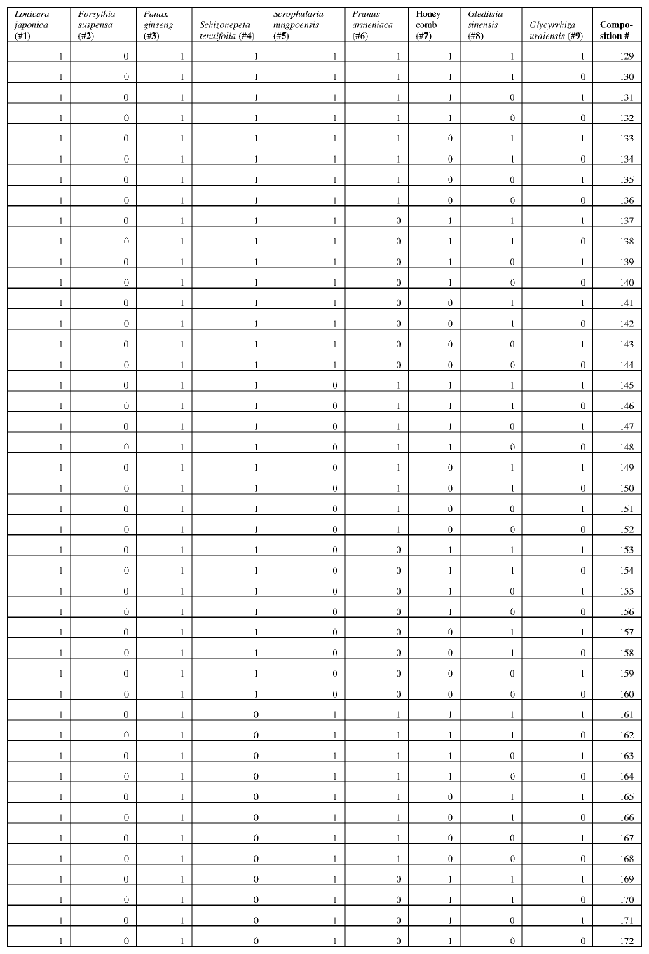

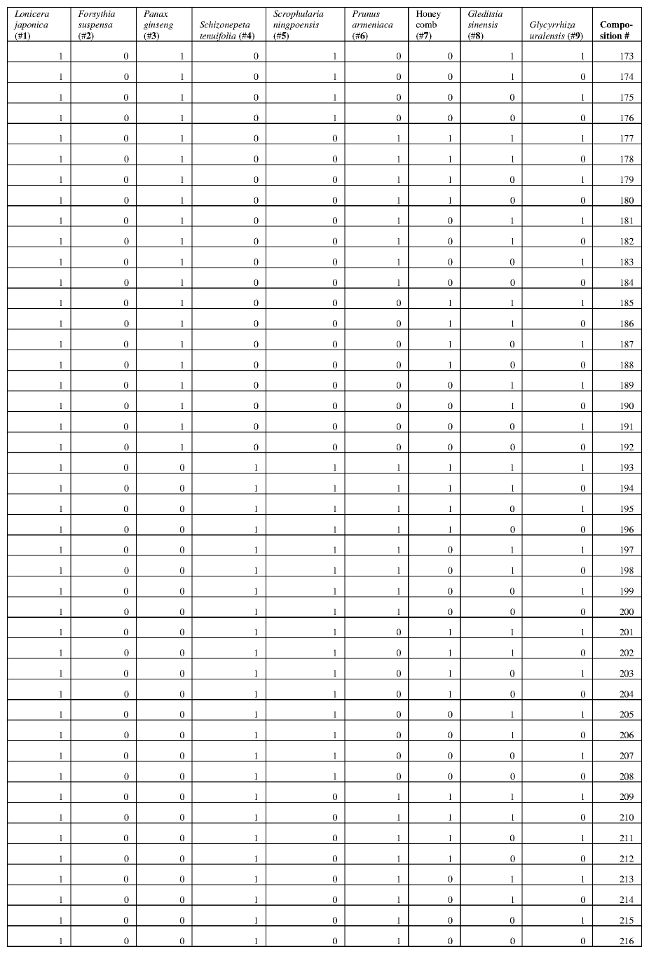

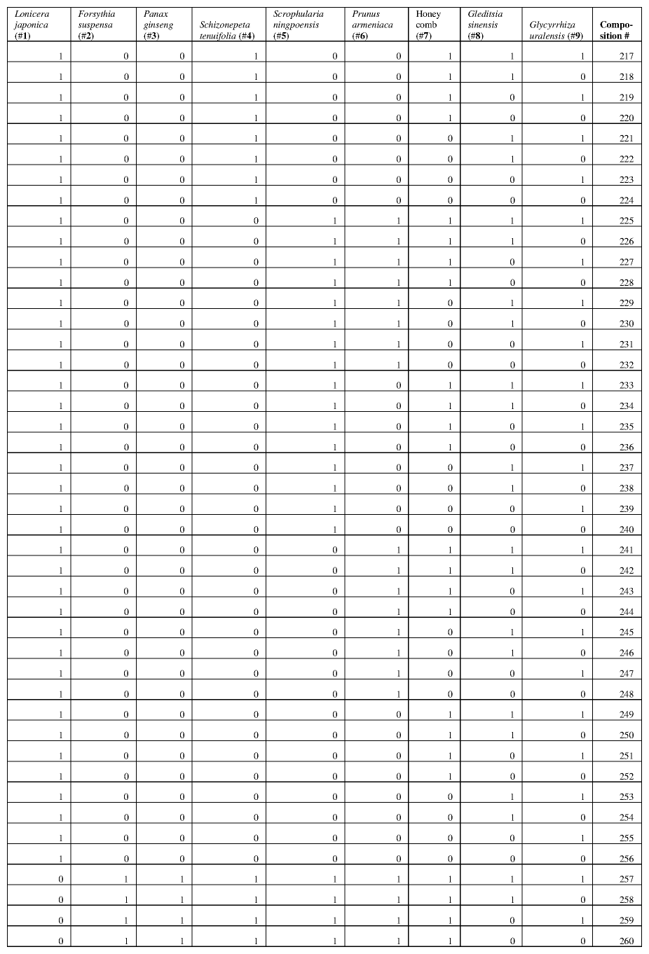

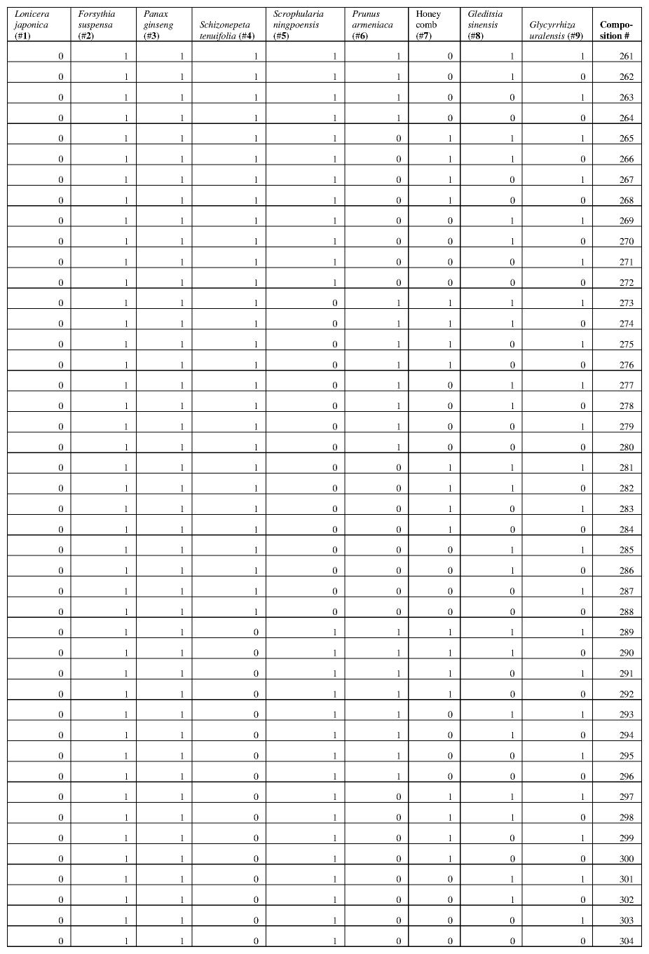

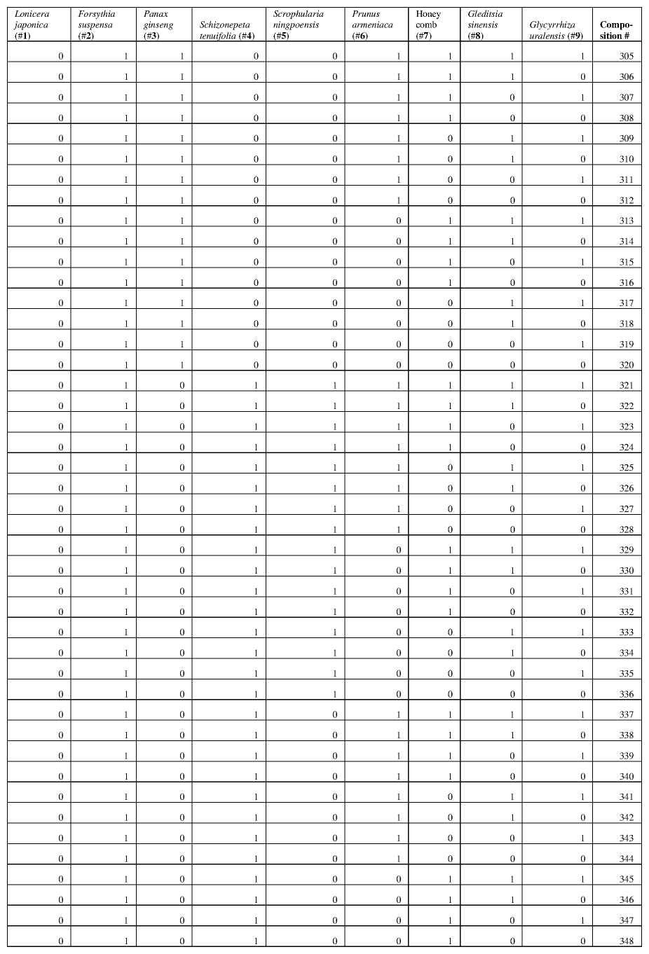

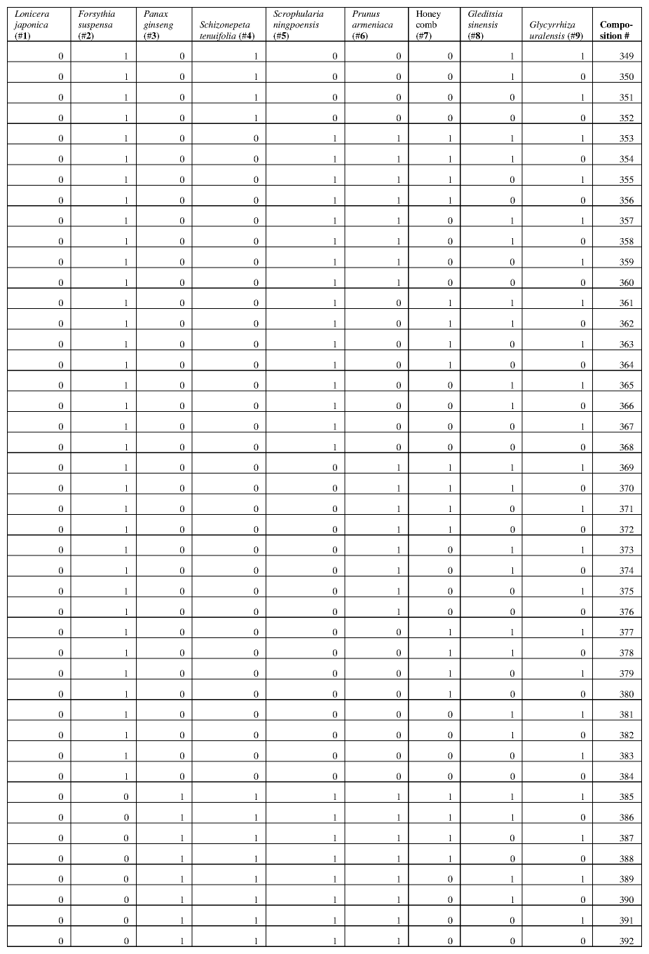

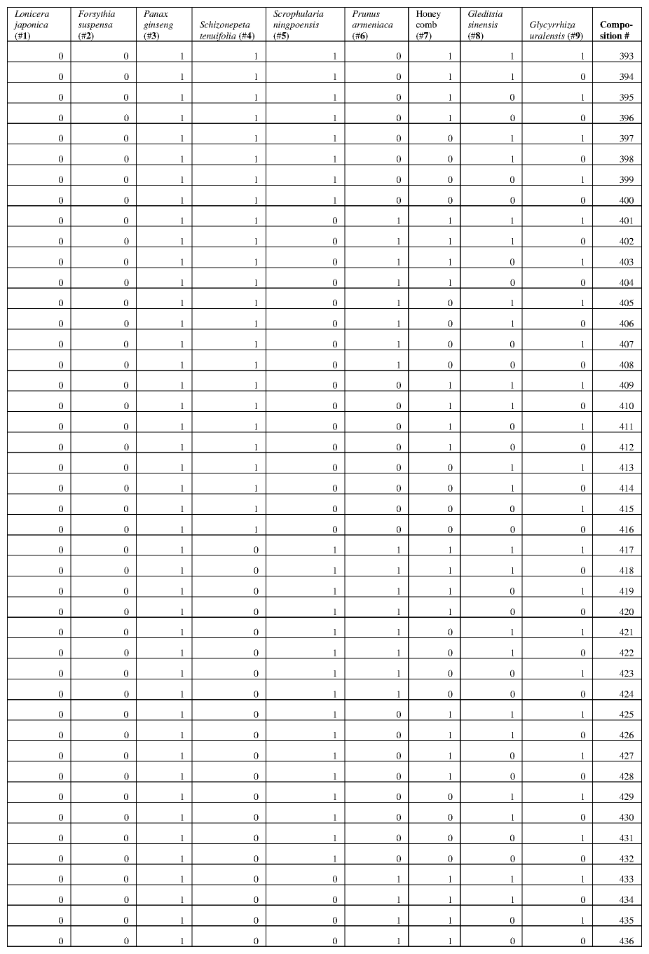

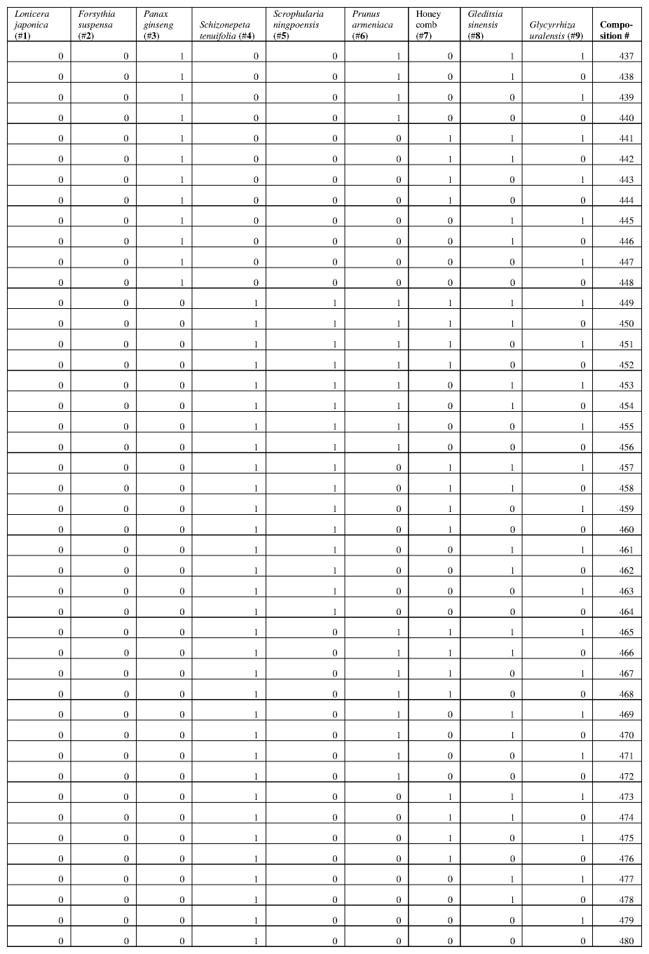

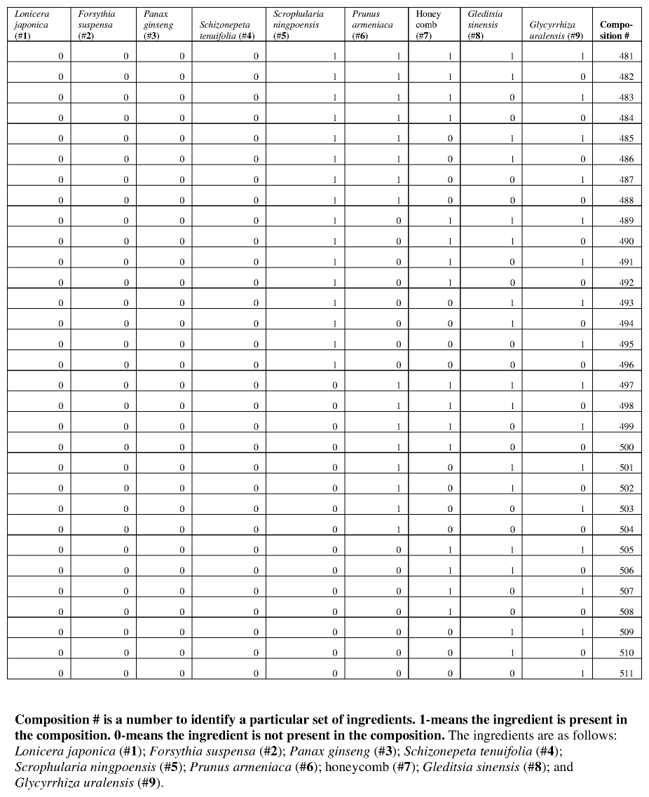

In some embodiments, the precursor composition comprises the combinations of TCM ingredients in Table 1 (FIG. 8). In table 1 (FIG. 8), the ingredients (top row) are as follows: Lonicera japonica (#1); Forsythia suspensa (#2); Panax ginseng (#3); Schizonepeta tenuifolia (#4); Scrophularia ningpoensis (#5); Prunus armeniaca (#6); honeycomb (#7); Gleditsia sinensis (#8); and Glycyrrhiza uralensis (#9). In the rows for a given composition #1-511 (right hand column), a “1” means the ingredient is present in the composition; and a “0” means the ingredient is not present in the composition.

Lonicera japonica (#1) is sometimes called Jin Yin Hua or Lonicerae Japonicae Flos or honeysuckle flower. In some embodiments, Lonicera japonica (#1) makes use of dried flower buds. In some embodiments, the Lonicera japonica (#1) is Lonicera japonica Thunb.

Forsythia suspensa (#2) is sometimes called Lian Qiao or Forsythiae Fructus. Forsythia suspensa (#2) makes use of dried fruit. In some embodiments, Forsythia suspensa (#2) is Forsythia suspensa (Thunb.) Vahl.

Panax ginseng (#3) is sometimes called Ren Shen or Ginseng Radix et Rhizoma. Panax ginseng (#3), in some embodiments, makes use of dried root or rhizome. In some embodiments, the Panax ginseng (#3) is Panax ginseng C. A. Mey.

Schizonepeta tenuifolia (#4) is sometimes called Jing Jie or Schizonepetae Herba or Japanese Catnip. Schizonepeta tenuifolia (#4), in some embodiments, makes use of the stem or bud. In some embodiments, Schizonepeta tenuifolia (#4) is Schizonepeta tenuifolia Briq.

Scrophularia ningpoensis (#5) is sometimes called Xaun Shen or Scrophulariae Radix. Scrophularia ningpoensis (#5), in some embodiments, makes use of dried root or rhizome. In some embodiments, Scrophularia ningpoensis (#5) is Scrophularia ningpoensis Hemsl.

Prunus armeniaca (#6), sometimes called Ku Xing Ren or Armeniacae Semen Amarum or apricot kernel. Prunus armeniaca (#6), in some embodiments, makes use of ripe seed or kernel. In some embodiments, Prunus armeniaca (#6) is Prunus armeniacavar. ansu Maxim.

Honeycomb (#7), sometimes called Feng Fang or Vespae Nidus, is of wasp nests. In some embodiments, the honeycomb (#7) is chosen from those of Polistes mandarinus Saussure, P. olivaceus, P. japonicus Saussure, and Parapolybia varia Fabricius. In any embodiment, unless otherwise specified, the honeycomb is choosable from Polistes mandarinus Saussure, P. olivaceus, P. japonicus Saussure, and Parapolybia varia Fabricius.

Gleditsia sinensis (#8) is sometimes called Zao Jiao Ci or Gleditsiae Spina. Gleditsia sinensis (#8), in some embodiments, makes use of thorns. In some embodiments, the Gleditsia sinensis (#8) is Gleditsia sinensis Lam.

Glycyrrhiza uralensis (#9) is sometimes called Gan Cao or Glycyrrhizae Radix et Rhizoma or Licorice. Glycyrrhiza uralensis (#9), in some embodiments, makes use of dried root or rhizome. In some embodiments, the Glycyrrhiza uralensis (#9) is Glycyrrhiza uralensis Fisch.

In some embodiments, the precursor composition comprises one of TCM ingredients in Table 1 (FIG. 8). In some embodiment, the precursor composition is composition #256, 384, 448, 480, 496, 504, 508, 510, or 511. In some embodiments, each of these precursor compositions has ingredients, which if present, are present in the relative amount shown in table 2 or table 3 or table 4 below.

In some embodiments, the precursor composition comprises two of TCM ingredients in Table 1 (FIG. 8). In some embodiments, the precursor composition is composition #128, 192, 224, 240, 248, 252, 254, 255, 320, 352, 368, 376, 380, 382, 383, 416, 432, 440, 444, 446, 447, 464, 472, 476, 478, 479, 488, 492, 494, 495, 500, 502, 503, 506, 507, or 509. In some embodiments, each of these precursor compositions has ingredients, which if present, are present in the relative amount shown in table 2 or table 3 or table 4 below.

In some embodiments, the precursor composition comprises three of TCM ingredients in Table 1 (FIG. 8). In some embodiments, the precursor composition is composition #64, 96, 112, 120, 124, 126, 127, 160, 176, 184, 188, 190, 191, 208, 216, 220, 222, 223, 232, 236, 238, 239, 244, 246, 247, 250, 251, 253, 288, 304, 312, 316, 318, 319, 336, 344, 348, 350, 351, 360, 364, 366, 367, 372, 374, 375, 378, 379, 381, 400, 408, 412, 414, 415, 424, 428, 430, 431, 436, 438, 439, 442, 443, 445, 456, 460, 462, 463, 468, 470, 471, 474, 475, 477, 484, 486, 487, 490, 491, 493, 498, 499, 501, or 505. In some embodiments, each of these precursor compositions has ingredients, which if present, are present in the relative amount shown in table 2 or table 3 or table 4 below.

In some embodiments, the precursor composition comprises four of TCM ingredients in Table 1 (FIG. 8). In some embodiments, the composition is composition #32, 48, 56, 60, 62, 63, 80, 88, 92, 94, 95, 104, 108, 110, 111, 116, 118, 119, 122, 123, 125, 144, 152, 156, 158, 159, 168, 172, 174, 175, 180, 182, 183, 186, 187, 189, 200, 204, 206, 207, 212, 214, 215, 218, 219, 221, 228, 230, 231, 234, 235, 237, 242, 243, 245, 249, 272, 280, 284, 286, 287, 296, 300, 302, 303, 308, 310, 311, 314, 315, 317, 328, 332, 334, 335, 340, 342, 343, 346, 347, 349, 356, 358, 359, 362, 363, 365, 370, 371, 373, 377, 392, 396, 398, 399, 404, 406, 407, 410, 411, 413, 420, 422, 423, 426, 427, 429, 434, 435, 437, 441, 452, 454, 455, 458, 459, 461, 466, 467, 469, 473, 482, 483, 485, 489, or 497. In some embodiments, each of these precursor compositions has ingredients, which if present, are present in the relative amount shown in table 2 or table 3 or table 4 below.

In some embodiments, the precursor composition comprises five of TCM ingredients in Table 1 (FIG. 8). In some embodiments, the composition is composition #16, 24, 28, 30, 31, 40, 44, 46, 47, 52, 54, 55, 58, 59, 61, 72, 76, 78, 79, 84, 86, 87, 90, 91, 93, 100, 102, 103, 106, 107, 109, 114, 115, 117, 121, 136, 140, 142, 143, 148, 150, 151, 154, 155, 157, 164, 166, 167, 170, 171, 173, 178, 179, 181, 185, 196, 198, 199, 202, 203, 205, 210, 211, 213, 217, 226, 227, 229, 233, 241, 264, 268, 270, 271, 276, 278, 279, 282, 283, 285, 292, 294, 295, 298, 299, 301, 306, 307, 309, 313, 324, 326, 327, 330, 331, 333, 338, 339, 341, 345, 354, 355, 357, 361, 369, 388, 390, 391, 394, 395, 397, 402, 403, 405, 409, 418, 419, 421, 425, 433, 450, 451, 453, 457, 465, or 481. In some embodiments, each of these precursor compositions has ingredients, which if present, are present in the relative amount shown in table 2 or table 3 or table 4 below.

In some embodiments, the precursor composition comprises six of TCM ingredients in Table 1 (FIG. 8). In some embodiments, the composition is composition #8, 12, 14, 15, 20, 22, 23, 26, 27, 29, 36, 38, 39, 42, 43, 45, 50, 51, 53, 57, 68, 70, 71, 74, 75, 77, 82, 83, 85, 89, 98, 99, 101, 105, 113, 132, 134, 135, 138, 139, 141, 146, 147, 149, 153, 162, 163, 165, 169, 177, 194, 195, 197, 201, 209, 225, 260, 262, 263, 266, 267, 269, 274, 275, 277, 281, 290, 291, 293, 297, 305, 322, 323, 325, 329, 337, 353, 386, 387, 389, 393, 401, 417, or 449. In some embodiments, each of these precursor compositions has ingredients, which if present, are present in the relative amount shown in table 2 or table 3 or table 4 below.

In some embodiments, the precursor composition comprises seven of TCM ingredients in Table 1 (FIG. 8). In some embodiments, the composition is composition #4, 6, 7, 10, 11, 13, 18, 19, 21, 25, 34, 35, 37, 41, 49, 66, 67, 69, 73, 81, 97, 130, 131, 133, 137, 145, 161, 193, 258, 259, 261, 265, 273, 289, 321, or 385. In some embodiments, each of these precursor compositions has ingredients, which if present, are present in the relative amount shown in table 2 or table 3 or table 4 below.

In some embodiments, the precursor composition comprises eight of TCM ingredients in Table 1 (FIG. 8). In some embodiments, the composition is composition #2, 3, 5, 9, 17, 33, 65, 129, or 257. In some embodiments, each of these precursor compositions has ingredients, which if present, are present in the relative amount shown in table 2 or table 3 or table 4 below.

In some embodiments, the precursor composition comprises ingredients #1, #2, and #4. In some embodiments, the precursor composition comprises ingredients #1, #2, and #4, and one, two or three ingredients from ingredients #3, #5, and #6. In some embodiments, the precursor composition comprises ingredients #1, #2, #3, and #4 or #1, #2, #3, #4, and #5 or #1, #2, #3, #4, and #6 or #1, #2, #4, and #5 or #1, #2, #4, #5, and #6 or #1, #2, #4, and #6.

In some embodiments, the precursor composition comprises two or more of the nine TCM ingredients present in an amount ranging from the lower amount to the higher amount in the following parts by weight in Table 2.

TABLE 2

| | Lonicera | Forsythia | Panax | Schizonepeta | Scrophularia | Prunus | | Gleditsia | Glycyrrhiza |
|---|---|---|---|---|---|---|---|---|---|
| | japonica | suspensa | ginseng | tenuifolia | ningpoensis | armeniaca | honeycomb | sinensis | uralensis |
| | Ingredient # | | | | | | | | |
| | 1 | 2 | 3 | 4 | 5 | 6 | 7 | 8 | 9 |
| Lower | 0.00 | 0.00 | 0.00 | 0.00 | 0.00 | 0.00 | 0.00 | 0.00 | 0.00 |
| Higher | 25.00 | 41.00 | 40.00 | 26.00 | 25.00 | 20.00 | 19.00 | 18.00 | 11.00 |

In some embodiments, the precursor composition comprises two or more of the nine TCM ingredients present in an amount ranging from the lower amount to the higher amount in the following parts by weight in Table 3.

TABLE 3

| | Lonicera | Forsythia | Panax | Schizonepeta | Scrophularia | Prunus | | Gleditsia | Glycyrrhiza |
|---|---|---|---|---|---|---|---|---|---|
| | japonica | suspensa | ginseng | tenuifolia | ningpoensis | armeniaca | honeycomb | sinensis | uralensis |
| | Ingredient # | | | | | | | | |
| | 1 | 2 | 3 | 4 | 5 | 6 | 7 | 8 | 9 |
| Lower | 0.00 | 0.00 | 0.00 | 0.00 | 0.00 | 0.00 | 0.00 | 0.00 | 0.00 |
| Higher | 5.00 | 19.00 | 20.00 | 4.00 | 5.00 | 1.00 | 1.50 | 2.00 | 1.00 |

In some embodiments, the precursor composition comprises two or more of the nine TCM ingredients, which if present, are present in an amount ranging from the lower amount to the higher amount in the following parts by weight in Table 4.

TABLE 4

| | Lonicera | Forsythia | Panax | Schizonepeta | Scrophularia | Prunus | | Gleditsia | Glycyrrhiza |
|---|---|---|---|---|---|---|---|---|---|
| | japonica | suspensa | ginseng | tenuifolia | ningpoensis | armeniaca | honeycomb | sinensis | uralensis |
| | Ingredient # | | | | | | | | |
| | 1 | 2 | 3 | 4 | 5 | 6 | 7 | 8 | 9 |
| Lower | 5.00 | 19.00 | 20.00 | 4.00 | 5.00 | 1.00 | 1.50 | 2.00 | 1.00 |
| Higher | 25.00 | 41.00 | 40.00 | 26.00 | 25.00 | 20.00 | 19.00 | 18.00 | 11.00 |

Making the Composition

In some embodiments, the composition comprises a decoction of the precursor composition. In some embodiments, the composition is in the form of a powder or paste made from the decoction whose supernatant aqueous phase was separated (from the residual part of the precursor composition) and was at least partially evaporated. In some embodiments, the composition is in the form of a powder made from the decoction whose supernatant aqueous phase was separated (from the residual part of the precursor composition) and was substantially or fully evaporated.

A method includes decocting the ingredients or the precursor composition. In some embodiments, the method includes combining the ingredients of the precursor composition and thereafter decocting the ingredients of the precursor composition.

In some embodiments, the precursor composition is boiled in an aqueous fluid optional comprising less than 50 v/v % an alcohol, such as ethanol, to form the composition. In some embodiments, the alcohol is present in the aqueous fluid at 0, 10, 20, 30, or 40% v/v %. In some embodiments, the aqueous fluid is water. In some embodiments, the precursor composition is boiled under pressure, that is, at a pressure that is 5-100% or 10-50% or 15-35% 760 mmHg. In some embodiments, the composition is in the form of a powder or paste made from the decoction whose supernatant aqueous phase was separated (from the residual part of the precursor composition) and was at least partially evaporated. In some embodiments, the composition is in the form of a powder made from the decoction whose supernatant aqueous phase was separated (from the residual part of the precursor composition) and was substantially or fully evaporated.

Composition

In some embodiments, the composition is made from a decoction of a precursor composition #1-511 in table 1 (FIG. 8). In some embodiments, each of these precursor compositions has ingredients, which, if present, are present in the relative amount shown in table 2 or table 3 or table 4. In some embodiments, the composition is in the form of a powder or powder made from the decoction whose supernatant aqueous phase was separated (from the residual part of the precursor composition) and was at least partially evaporated.

In some embodiments, the composition is made from a decoction of a precursor composition which comprises the listed ingredients in FIG. 4. In some embodiments, each of these precursor compositions has ingredients, which, if present, are present in the relative amount shown in table 2 or table 3 or table 4. In some embodiments, the composition is in the form of a powder or powder made from the decoction whose supernatant aqueous phase was separated (from the residual part of the precursor composition) and was at least partially evaporated.

In some embodiments, the composition is made from a decoction of a precursor composition which comprises the ingredients of precursor composition #1. In some embodiments, precursor composition #1 has ingredients present in a relative amount less than the maximum amount shown in table 2 or table 3. In some embodiments, precursor composition #1 has ingredients present in the relative amount shown in table 4. In some embodiments, the composition is in the form of a powder or powder made from the decoction whose supernatant aqueous phase was separated (from the residual part of the precursor composition) and was at least partially evaporated.

In some embodiments, the composition is made from a decoction of a precursor composition which comprises one of TCM ingredients in Table 1 (FIG. 8). In some embodiments, the composition is made from a decoction of a precursor composition which comprises the ingredients of precursor composition #256, 384, 448, 480, 496, 504, 508, 510, or 511. In some embodiments, each of these precursor compositions has ingredients, which, if present, are present in the relative amount shown in table 2 or table 3 or table 4. In some embodiments, the composition is in the form of a powder or powder made from the decoction whose supernatant aqueous phase was separated (from the residual part of the precursor composition) and was at least partially evaporated.

In some embodiments, the composition is made from a decoction of a precursor composition which comprises the ingredients of precursor composition #128, 192, 224, 240, 248, 252, 254, 255, 320, 352, 368, 376, 380, 382, 383, 416, 432, 440, 444, 446, 447, 464, 472, 476, 478, 479, 488, 492, 494, 495, 500, 502, 503, 506, 507, or 509. In some embodiments, each of these precursor compositions has ingredients, which, if present, are present in the relative amount shown in table 2 or table 3 or table 4. In some embodiments, the composition is in the form of a powder or powder made from the decoction whose supernatant aqueous phase was separated (from the residual part of the precursor composition) and was at least partially evaporated.

In some embodiments, the composition is made from a decoction of a precursor composition which comprises the ingredients of precursor composition #64, 96, 112, 120, 124, 126, 127, 160, 176, 184, 188, 190, 191, 208, 216, 220, 222, 223, 232, 236, 238, 239, 244, 246, 247, 250, 251, 253, 288, 304, 312, 316, 318, 319, 336, 344, 348, 350, 351, 360, 364, 366, 367, 372, 374, 375, 378, 379, 381, 400, 408, 412, 414, 415, 424, 428, 430, 431, 436, 438, 439, 442, 443, 445, 456, 460, 462, 463, 468, 470, 471, 474, 475, 477, 484, 486, 487, 490, 491, 493, 498, 499, 501, or 505. In some embodiments, each of these precursor compositions has ingredients, which, if present, are present in the relative amount shown in table 2 or table 3 or table 4. In some embodiments, the composition is in the form of a powder or powder made from the decoction whose supernatant aqueous phase was separated (from the residual part of the precursor composition) and was at least partially evaporated.

In some embodiments, the composition is made from a decoction of a precursor composition which comprises the ingredients of precursor composition #32, 48, 56, 60, 62, 63, 80, 88, 92, 94, 95, 104, 108, 110, 111, 116, 118, 119, 122, 123, 125, 144, 152, 156, 158, 159, 168, 172, 174, 175, 180, 182, 183, 186, 187, 189, 200, 204, 206, 207, 212, 214, 215, 218, 219, 221, 228, 230, 231, 234, 235, 237, 242, 243, 245, 249, 272, 280, 284, 286, 287, 296, 300, 302, 303, 308, 310, 311, 314, 315, 317, 328, 332, 334, 335, 340, 342, 343, 346, 347, 349, 356, 358, 359, 362, 363, 365, 370, 371, 373, 377, 392, 396, 398, 399, 404, 406, 407, 410, 411, 413, 420, 422, 423, 426, 427, 429, 434, 435, 437, 441, 452, 454, 455, 458, 459, 461, 466, 467, 469, 473, 482, 483, 485, 489, or 497. In some embodiments, each of these precursor compositions has ingredients, which, if present, are present in the relative amount shown in table 2 or table 3 or table 4. In some embodiments, the composition is in the form of a powder or powder made from the decoction whose supernatant aqueous phase was separated (from the residual part of the precursor composition) and was at least partially evaporated.

In some embodiments, the composition is made from a decoction of a precursor composition which comprises the ingredients of precursor composition #16, 24, 28, 30, 31, 40, 44, 46, 47, 52, 54, 55, 58, 59, 61, 72, 76, 78, 79, 84, 86, 87, 90, 91, 93, 100, 102, 103, 106, 107, 109, 114, 115, 117, 121, 136, 140, 142, 143, 148, 150, 151, 154, 155, 157, 164, 166, 167, 170, 171, 173, 178, 179, 181, 185, 196, 198, 199, 202, 203, 205, 210, 211, 213, 217, 226, 227, 229, 233, 241, 264, 268, 270, 271, 276, 278, 279, 282, 283, 285, 292, 294, 295, 298, 299, 301, 306, 307, 309, 313, 324, 326, 327, 330, 331, 333, 338, 339, 341, 345, 354, 355, 357, 361, 369, 388, 390, 391, 394, 395, 397, 402, 403, 405, 409, 418, 419, 421, 425, 433, 450, 451, 453, 457, 465, or 481. In some embodiments, each of these precursor compositions has ingredients, which, if present, are present in the relative amount shown in table 2 or table 3 or table 4. In some embodiments, the composition is in the form of a powder or powder made from the decoction whose supernatant aqueous phase was separated (from the residual part of the precursor composition) and was at least partially evaporated.

In some embodiments, the composition is made from a decoction of a precursor composition which comprises the ingredients of precursor composition #8, 12, 14, 15, 20, 22, 23, 26, 27, 29, 36, 38, 39, 42, 43, 45, 50, 51, 53, 57, 68, 70, 71, 74, 75, 77, 82, 83, 85, 89, 98, 99, 101, 105, 113, 132, 134, 135, 138, 139, 141, 146, 147, 149, 153, 162, 163, 165, 169, 177, 194, 195, 197, 201, 209, 225, 260, 262, 263, 266, 267, 269, 274, 275, 277, 281, 290, 291, 293, 297, 305, 322, 323, 325, 329, 337, 353, 386, 387, 389, 393, 401, 417, or 449. In some embodiments, each of these precursor compositions has ingredients, which, if present, are present in the relative amount shown in table 2 or table 3 or table 4. In some embodiments, the composition is in the form of a powder or powder made from the decoction whose supernatant aqueous phase was separated (from the residual part of the precursor composition) and was at least partially evaporated.

In some embodiments, the composition is made from a decoction of a precursor composition which comprises the ingredients of precursor composition #4, 6, 7, 10, 11, 13, 18, 19, 21, 25, 34, 35, 37, 41, 49, 66, 67, 69, 73, 81, 97, 130, 131, 133, 137, 145, 161, 193, 258, 259, 261, 265, 273, 289, 321, or 385. In some embodiments, each of these precursor compositions has ingredients, which, if present, are present in the relative amount shown in table 2 or table 3 or table 4. In some embodiments, the composition is in the form of a powder or powder made from the decoction whose supernatant aqueous phase was separated (from the residual part of the precursor composition) and was at least partially evaporated.

In some embodiments, the composition is made from a decoction of a precursor composition which comprises the ingredients of precursor composition #2, 3, 5, 9, 17, 33, 65, 129, or 257. In some embodiments, each of these precursor compositions has ingredients, which, if present, are present in the relative amount shown in table 2 or table 3 or table 4. In some embodiments, the composition is in the form of a powder or powder made from the decoction whose supernatant aqueous phase was separated (from the residual part of the precursor composition) and was at least partially evaporated.

In some embodiments, the composition is made from a decoction of a precursor composition which comprises the ingredients of a precursor composition comprising ingredients #1, #2, and #4. In some embodiments, the composition is made from a decoction of a precursor composition which comprises the ingredients of a precursor composition comprising Ingredients #1, #2, and #4, and one, two or three ingredients from #3, #5, and #6. In some embodiments, the composition is made from a decoction of a precursor composition which comprises the ingredients of a precursor composition comprising Ingredients #1, #2, #3, and #4 or #1, #2, #3, #4, and #5 or #1, #2, #3, #4, and #6 or #1, #2, #4, and #5 or #1, #2, #4, #5, and #6 or #1, #2, #4, and #6. In some embodiments, each of these precursor compositions has ingredients, which, if present, are present in the relative amount shown in table 2 or table 3 or table 4. In some embodiments, the composition is in the form of a powder or powder made from the decoction whose supernatant aqueous phase was separated (from the residual part of the precursor composition) and was at least partially evaporated.

In some embodiments, the composition further comprises a pharmaceutically or dietary acceptable excipient. In some embodiments, the pharmaceutically or dietary acceptable excipient is chosen from solvents, binders, lubricants, herbal carriers, oils and salts.

In some embodiments, the composition comprises water, juice or glycerin. In some embodiments, the juice is elderberry juice, orange juice or apple juice. In some embodiments, the composition comprises water, elderberry juice, and glycerin.

In some embodiments, the composition is made from a decoction of a precursor composition which comprises the listed ingredients in FIG. 4 and further comprises one or more pharmaceutically or dietary acceptable excipients.

In some embodiments, the composition disclosed herein may be formulated in various dosage forms suitable for oral administration. In some embodiments, the composition is in the form chosen from tablets, pellets, lozenges, granules, capsules, solutions, emulsions, and suspensions. In some embodiments, the composition is in the form of a tablet, such as a 100-500 mg tablet. In some embodiments, the composition is in the form of a tincture. In some embodiments, the composition is in the form of a dietary supplement.

In some embodiments, the composition is in the form of a gel, emulsion, lotion or cream. In some embodiments, the composition is in the form of a gel, emulsion, or sprayable composition.

Treatment

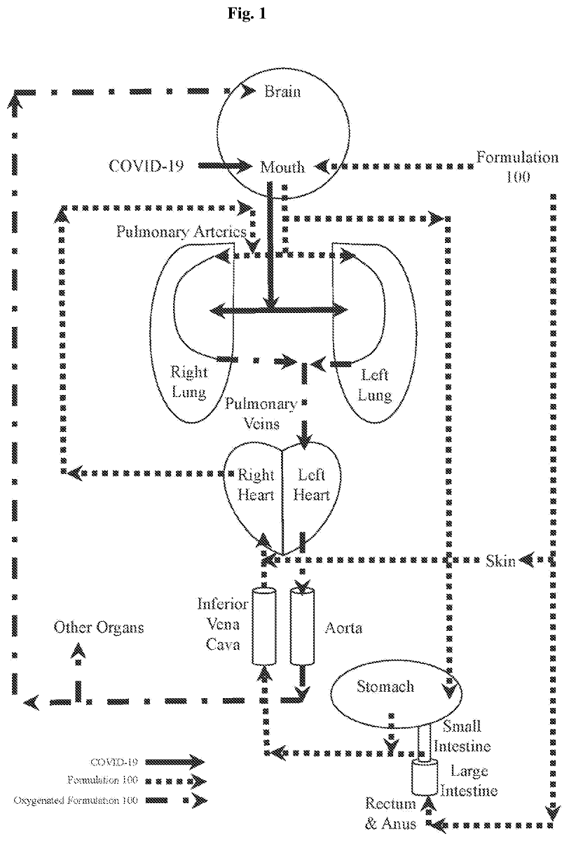

Turning now to FIG. 1, the formulation 100 may be administered to the body orally, nasally, anally, or through the skin. When administered to the skin, the formulation 100 may be absorbed by the skin and enter the blood stream to travel to organs within the body. When administered orally, the formulation 100 may go to the stomach or small and large intestine before entering the blood stream. When administered nasally, the formulation may be in the form of an aerosol that is sprayed in the nose or inhaled. When administered anally, the formulation may be inserted in the anus through the rectum and into the large intestine. The formulation may then be absorbed into the blood stream. Once in the bloodstream through any desired manner, the formulation may then travel through the right side of the heart and then to the pulmonary arteries in the lungs. When administered orally, the formulation may also be absorbed into blood vessels in the mouth and throat and reach the pulmonary arteries in the lungs. In the lungs, the formulation may assist the lungs with detoxification in order to better fend off external attacks. From the lungs, the formulation may travel through the left side of the heart and reach the brain and other internal organs.

In an exemplary embodiment, the formulation 100 may take the form of a beverage. The beverage could be consumed four ounces at a time. The formulation 100 may be consumed twice a day for 15 days. The formulation 100 may optionally be taken with or immediately before orange juice. The orange juice may improve absorption, and add a sweetness to the earthy flavor.

In another exemplary embodiment, the formulation may take the form of an anal suppository. The formulation may be inserted into the anus and absorbed into the blood vessels for rapid absorption into the body where it can aid the resistance to COVID-19.

In another exemplary embodiment, the formulation may be administered as a vapor or aerosol. The formulation may then be breathed in with normal airflow directly to the lungs. The vapor formulation may then provide support to the lungs directly in order to resist COVID-19.

In another exemplary embodiment, the formulation may be in a cream, ointment, oil, or other topical form. The formulation may be spread onto the skin where it is absorbed into the bloodstream to support the body's resistance to COVID-19. The formulation may optimally be rubbed near the lungs to provide faster access to the lungs for treatment and aid in resisting COVID-19.

Treatment of Coronavirus Infection

A method treats a patient in need of treatment for coronavirus infection by administering to the patient an effective amount of the composition disclosed herein. In some embodiments, the coronavirus infection is a SARS-CoV infection. In some embodiments, the coronavirus infection is from a SARS-CoV-2 infection. In some embodiments, the coronavirus infection is from COVID-19. In some embodiments, the patients are human.

As noted in the examples, experiments confirmed that RDS has anti-corona virus activity and determined RDS's anti-SARS-CoV activity or anti-SARS-CoV-2 activity and its half maximal inhibition dosage (IC₅₀).

For experimental verification, RDS was be serially diluted into: 1, 1:2, 1:4, 1:8, 1:16, & 1:32 with cell culture medium (DMEM). Vero E6 (example 1) or A549(ACE2) (Example 2) cells were treated with RDS for 1 hour, and then infected with SARS-CoV (GFP) pseudovirus for 6 or 4 hours. Following infection, inhibition of viral replication was analyzed by flow cytometer for GFP expression, and IC₅₀ values were determined.

In the examples, CoV-2-PIE™ from Virongy is a viral infection enhancer designed specifically to facilitate coronavirus and pseudovirus infection of target cells. CoV-2-PIE™ is designed to facilitate the infection of a variety of host cells by coronaviruses and pseudoviruses. CoV-2-PIE™ can enhance viral infection rates by 5 to 20-fold.

In the examples, SARS-CoV-GFP is a lentivirus-based pseudovirus carrying the S gene (Spike protein gene) of SARS-CoV virus. The pseudovirus infects cells as SARS-CoV, and uses the receptor ACE2 to infect cells. But inside the pseudovirus, the genome is a GFP reporter gene rather than the SARS-CoV genome. The pseudovirus can infect a cell as SARS-CoV but cannot replicate. Thus, it is commonly used for studying SARS-CoV virus entry without the need for a BL3 lab.

Currently, Virongy is selling SARS-CoV (GFP) virus for research use. The study was performed using Virongy's SARS-CoV (GFP) pseudovirus.

Treatment of Flu Infection

The composition may be used to treat a patient in need of treatment for a viral infection, e.g., the flu, including but not limited to Influenza A (“fluA”), by administering to the patient an effective amount of the composition. In some embodiments, the patients are human.

Disinfectant

The present composition makes it possible to clean and/or disinfect surfaces of animate and inanimate objects.

In some embodiments, the method of disinfecting an animate object includes applying an effective amount of the composition to a topical surface of an animate object. In some embodiments, the composition is in the form of a gel, emulsion, lotion or cream. In some embodiments, the animate object is a human. In some embodiments, the surface is skin. In some embodiments, the skin is on a human hand.

In some embodiments, the method of disinfecting an inanimate object includes applying an effective amount of the composition to a topical surface of an inanimate object. In some embodiments, the composition is in the form of a gel, emulsion, or a sprayable composition. In some embodiments, the inanimate object is chosen from tables, hard-backed chairs, doorknobs, light switches, phones, tablets, touch screens, remote controls, keyboards, handles, desks, toilets, and sinks. In some embodiments, the composition is applied to a surface of an inanimate object. In some embodiments, the inanimate object is in a household setting. In some embodiments, the inanimate object is in an industrial or business setting. In some embodiments, the inanimate object is in a medical setting.

### Example 1

A composition (“RDS”) is a decoction of Lonicera japonica; Forsythia suspensa; Panax ginseng; Schizonepeta tenuifolia; Scrophularia ningpoensis; Prunus armeniaca; honeycomb; Gleditsia sinensis; and Glycyrrhiza uralensis. See FIG. 4 for a listing of ingredients.

### Example 2

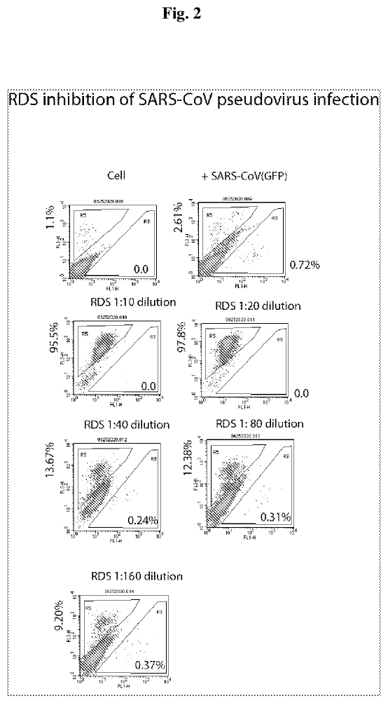

The following steps were performed to generate data in FIG. 2.

1) Seeding 1×10⁵ Vero-E6 cells per well in a 12-well cell culture plates in 1 ml culture medium. Grow cell overnight at 37° C.

2) The next day, move medium from each well of the 12 well plate. Add 300 μl fresh culture medium,

3) Add 30 μl diluted RDS to treat cells for 30 minutes at 37° C.

RDS dilution
A—NO dilution
B—1:1 dilution, take 1 ml A+1 ml culture medium
C—1:2 dilution, take 1 ml B+1 ml culture medium
D—1:4 dilution, take 1 ml C+1 ml culture medium
E—1:8 dilution, take 1 ml D+1 ml culture medium
F—1:16 dilution, take 1 ml E+1 ml culture medium

4) Add 33 ul Virongy CoV-2-PIE™ (Described above) to treat cells for other 30 minutes at 37° C.

5) Add 100 μl 60× concentrated SARS-CoV (GFP) virus, add 10% (V/V) Virongy CoV-2-PIE™.

6) Incubate for 4 hours.

7) Add 1 ml fresh culture medium with RDS added in concentration as listed above.

8) Analyze cellular GFP expression by flow cytometry at 48 hours post infection.

FIG. 1. VeroE6 cells were treated with serially diluted RDS, and infected with SARS-CoV-GFP pseudovirus, and inhibition of viral infection was quantified at 48 hours post infection by flow cytometry. ‘Uninfected Cell’ and ‘SARS-CoV-GFP-infected but RDS-untreated cells’ were used as controls (top right and left panel).

Conclusion: RDS inhibits SARS-CoV (GFP) viral infection of VeroE6 cells with a IC₅₀ around 1:160 diluted concentration.

### Example 3

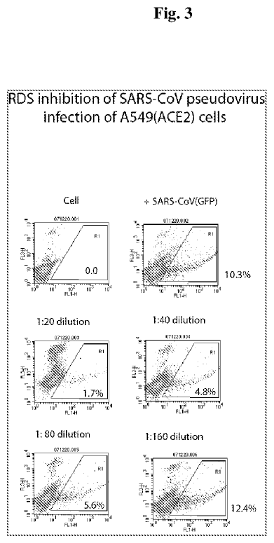

The following steps were performed to generate data in FIG. 3.

1) Seeding 1×10⁵ A549(ACE2) cells per well in a 12-well cell culture plates in 1 ml culture medium. Grow cell overnight at 37° C.

2) The next day, move medium from each well of the 12 well plate. Add 300 μl fresh culture medium.

3) Add 30 μl diluted RDS to treat cells for 30 minutes at 37° C.

RDS dilution
A—NO dilution
B—1:1 dilution, take 1 ml A+1 ml culture medium
C—1:2 dilution, take 1 ml B+1 ml culture medium
D—1:4 dilution, take 1 ml C+1 ml culture medium
E—1:8 dilution, take 1 ml D+1 ml culture medium
F— 1:16 dilution, take 1 ml E+1 ml culture medium

4) Add 33 ul Virongy CoV-2-PIE™ to treat cells for other 30 minutes at 37° C.

5) Add 100 μl 60× concentrated SARS-CoV (GFP) virus, add 10% (V/V) CoV-2-PIE™.

6) Incubate for 4 hours.

7) After infection, cells were washed twice to remove virus and RDS.

8) Add 1 ml fresh culture medium without RDS, culture for 48 hours

9) Analyze cellular GFP expression by flow cytometry at 48 hours post infection.

FIG. 2. A549(ACE2) cells were treated with serially diluted RDS, and infected with SARS-CoV-GFP pseudovirus for 4 hours. Following infection, RDS was washed away and inhibition of viral infection was quantified at 48 hours post infection by flow cytometry. Uninfected Cell and SARS-CoV-GFP-infected but RDS-untreated cells were used as controls (top right and left panel).

Conclusion: RDS inhibits SARS-CoV (GFP) viral infection of A549 (ACE2) cells with a IC₅₀ around 1:80 diluted concentration.

### Example 3

The composition of Example 1 was studied for the anti-SARS-CoV-2 activity.

### SUMMARY

The study tested the anti-SARS-CoV-2 activity in the composition of Example 1, (RDS (Respiratory Detox Support)), using VIRONGY LLC's SARS-CoV-2 S protein pseudotyped GFP and luciferase reporter lentiviruses. The target cells were Vero E6 and ACE2-expressing A549 cell (human lung epithelia cell). Experiments tested and quantified the anti-SARS-CoV-2 activity of RDS, and found that RDS possesses anti-SARS-CoV-2 activity. It was discovered that:

RDS possesses anti-SARS-CoV-2 activity. The IC₅₀ (50% virus inhibition dosage) of RDS was determined to be at 1:230-fold dilution for the inhibition of viral infection of Vero E6 cells.

RDS inhibited over 90% SARS-CoV-2 viral entry into Vero E6 cells at 1:40 dilution.

Mechanistic studies using SARS-CoV-2 pseudovirus infection of A549(ACE2) cells further confirmed that RDS blocked SARS-CoV S protein-mediated viral entry into ACE2+ target cells.

Introduction

Experiments determined whether RDS has anti-coronavirus activity and quantified its antiviral potency. For the experiments, RDS was serially diluted, and then used to treat Vero E6 or A549(ACE2) target cells to block SARS-CoV-2 pseudovirus infection. Inhibition of viral transduction and viral entry were analyzed using reporter GFP (green fluorescent protein) or Luc (luciferase) expression. The IC₅₀ inhibition dosage was also determined.

Materials and Methods

Viruses: SARS-CoV-2(GFP) and SARS-CoV-2(Luc) reporter pseudoviruses were assembled by VIRONGY LLC using in-house lentiviral assembly system. The infectivity was quantified by infecting in-house A549(ACE2) cells.

Cell culture medium: Cells were maintained in Dulbecco-modified Eagle's medium (DMEM) (Invitrogen) containing 10% heat-inactivated FBS and 1× penicillin-streptomycin (Invitrogen).

luciferase assay: Cells were lysed using Luciferase Assay Lysis Buffer (Promega) Luminescence was measured by using GloMax® Discover Microplate Reader (Promega).

### Experimental Procedure

A. Quantification of the Anti-Viral Activity of RDS Using SARS-CoV-2(Luc) Pseudovirus

Seeded were 1×10⁵ Vero E6 cells per well in 12-well cell culture plates in 1 ml culture medium. Grown were cells overnight at 37° C.

The next day, the medium was removed from each well of the 12 well plates. 300 μl of fresh were added to each well of the 12-well plate.

30 μl of diluted RDS were added to the culture medium to treat cells for 30 minutes at 37° C.

RDS dilution

A—NO dilution

B—1:1 dilution, take 1 ml A+1 ml culture medium

C—1:2 dilution, take 1 ml B+1 ml culture medium

D—1:4 dilution, take 1 ml C+1 ml culture medium

E—1:8 dilution, take 1 ml D+1 ml culture medium

F—1:16 dilution, take 1 ml E+1 ml culture medium

G—1:32 dilution, take 1 ml F+1 ml culture medium

33 μl of VIRONGY brand CoV-2-PIE were added to the medium to treat cells for another 30 minutes at 37° C.

100 μl of concentrated SARS-CoV-2(Luc) virus were added to the medium, and then 10% (V/V) of CoV-2-PIE were added to the medium.

Cells were infected for 6 hours.

After infection, cells were washed twice to remove virus and RDS.

1 ml of fresh culture medium without RDS were added, and the resultant medium was cultured for 72 hours.

Luc expression was analyzed by luciferase assay at 72 hours post infection.

### Results

As shown in FIG. 5, RDS inhibited SARS-CoV-2(Luc) viral infection of Vero E6 cells, and the IC50 was at 1:230-fold dilution.

### Results

As shown in FIG. 5, RDS inhibited SARS-CoV-2(Luc) viral infection of Vero E6 cells, and the IC₅₀ was at 1:230-fold dilution.

FIG. 5 shows RDS dosage-dependent inhibition of SARS-CoV-2(Luc) pseudovirus. Vero E6 cells were treated with serially diluted RDS, and infected with SARS-CoV-2(Luc) pseudovirus for 6 hours. Cells were washed to remove the virus and RDS, and cultured in the absence of RDS. Inhibition of viral infection was quantified at 72 hours post infection by luciferase assay. Uninfected Cell and SARS-CoV-2-Luc-infected but RDS-untreated cells were used as controls. The assay was performed in triplicates. The dose-response curve was plotted, and the IC₅₀ of RDS was quantified to be at 1:230 dilution (right panel).

B. Testing Effects of RDS on the Entry of SARS-CoV-2(GFP) Pseudovirus

Seeded were 1×10⁵ A549(ACE2) cells per well in 12-well cell culture plates in 1 ml culture medium. Grown were cells overnight at 37° C.

The next day, the medium was removed from each well of the 12 well plates. The removed medium was added to 300 μl fresh culture medium.

30 μl of diluted RDS were added to treat cells for 30 minutes at 37° C.

RDS dilution

A—NO dilution

B—1:1 dilution, take 1 ml A+1 ml culture medium

C—1:2 dilution, take 1 ml B+1 ml culture medium

D—1:4 dilution, take 1 ml C+1 ml culture medium

E—1:8 dilution, take 1 ml D+1 ml culture medium

F—1:16 dilution, take 1 ml E+1 ml culture medium

G—1:32 dilution, take 1 ml F+1 ml culture medium

100 μl of concentrated SARS-CoV-2(GFP) virus were added to the medium.

The medium was incubated for 6 hours.

After infection, cells were washed twice to remove virus and RDS.

1 ml of fresh culture medium without RDS were added and then cultured for 48 hours.

Cellular GFP expression was analyzed by flow cytometry at 48 hours post infection.

### Results

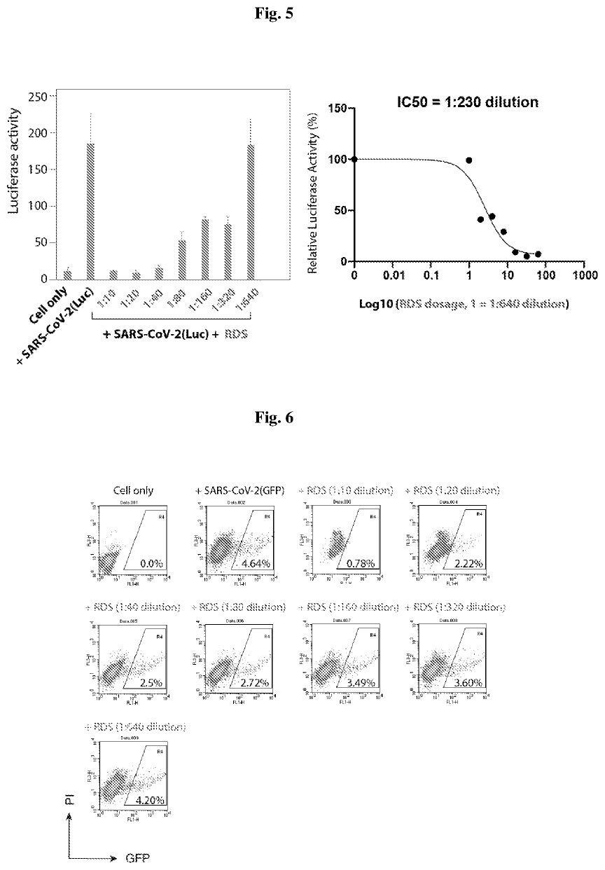

As shown in FIG. 6, RDS inhibited SARS-CoV-2(GFP) infection of A549(ACE2) cells. For the experiment, RDS was used only during viral infection for 6 hours, and was washed away with the virus after infection. Cells were cultured without RDS following infection. For flow cytometry analyses, propidium iodide (PI) was added to stain for dead cells. GFP+ cells were analyzed only in live cell population to exclude non-specific effects of cytotoxicity. This experiment demonstrated that RDS blocked viral early infection processes, likely viral entry.

### Example 4

The Study of the Anti-Flu a Virus Activity of RDS

### SUMMARY

The effects of RDS for the inhibition of FluA virus infection of MDCK cells were tested. Infectious FluA (GFP) reporter virus was assembled and used to infect MDCK cells in the presence or absence of RDS (composition of example 1), which were serially diluted (1:10, 1:20, 1:40, 1:80, 1:160, 1:320). Inhibition of viral infection was quantified at 36 hours post infection using flow cytometry. To monitor cytotoxicity of RDS, propidium iodide (PI) was added to stain for dead cells. GFP+ cells were analyzed only in live cell population to exclude non-specific cytotoxic effects. This experiment demonstrated that RDS completely blocked viral infection at dilutions from 1:10 to 1:80, and partially inhibited FluA at 1:160 and 1:320 dilution.

Introduction

This experiment determined whether RDS has anti-Flu A activity and quantified its antiviral potency. For the experiments, RDS was serially diluted, and then used to treat MDCK cells to block Flu A infection. Inhibition of viral infection were analyzed using reporter GFP (green fluorescent protein).

Materials and Methods

Viruses: Flu A(GFP) reporter virus were assembled by Virongy LLC. The infectivity was quantified by infecting human MDCK cells.

Cell culture medium: Cells were maintained in Dulbecco-modified Eagle's medium (DMEM) (Invitrogen) containing 10% heat-inactivated FBS and 1× penicillin-streptomycin (Invitrogen).

### Experimental Procedure

Quantification of the Anti-Viral Activity of RDS Using FluA (GFP) Reporter Virus

Seeded were 1×10⁵ MECK cells per well in 12-well cell culture plates in 1 ml culture medium. Grown were cells overnight at 37° C.

The next day, the medium was removed from each well of the 12 well plates. 300 μl of fresh culture medium were added to each well of the 12-well plate.

30 μl of diluted RDS were added to the medium to treat cells for 30 minutes at 37° C.

RDS dilution

A—NO dilution

B—1:1 dilution, take 1 ml A+1 ml culture medium

C—1:2 dilution, take 1 ml B+1 ml culture medium

D—1:4 dilution, take 1 ml C+1 ml culture medium

E—1:8 dilution, take 1 ml D+1 ml culture medium

F—1:16 dilution, take 1 ml E+1 ml culture medium

G—1:32 dilution, take 1 ml F+1 ml culture medium

500 μl of FluA (GFP) virus were added to the medium, and then 10% (V/V) RDS were added to the medium.

The cells were infected for 6 hours.

After infection, the supernatant was removed.

1 ml of fresh culture medium with RDS were added to the removed medium and then cultured for 36 hours.

GFP expression was analyzed at 36 hours post infection by flow cytometer.

### Results

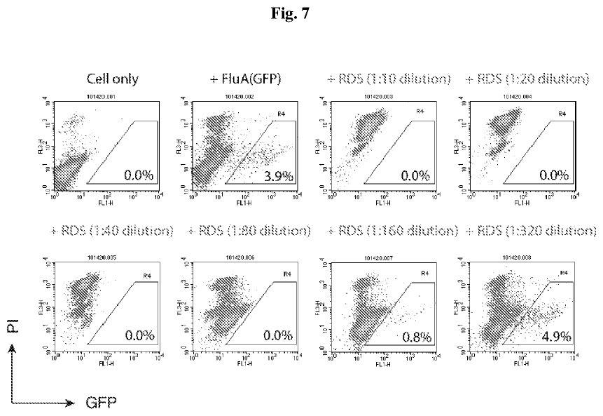

As shown in FIG. 7, RDS inhibited FluA infection of MDCK cells. For the experiment, RDS was used during infection for 6 hours. Infected cells were cultured with RDS. For flow cytometry analyses, propidium iodide (PI) was added to stain for dead cells. GFP+ cells were analyzed only in live cell population to exclude non-specific effects of cytotoxicity. This experiment demonstrated that RDS blocked FluA (GFP) virus infection.

### Example 5

Precursor compositions A-N comprises two or more of the nine TCM ingredients present in an amount shown in the row of the examples in the following parts by weight in Table 5.

TABLE 5

| | | | | | | | Polistes | | |
|---|---|---|---|---|---|---|---|---|---|
| | Lonicera | Forsythia | Panax | Schizonepeta | Scrophularia | Prunus | mandarinus | Gleditsia | Glycyrrhiza |
| | japonica | suspensa | ginseng | tenuifolia | ningpoensis | armeniaca | Saussure | sinensis | uralensis |
| | Ingredient # | | | | | | | | |
| | 1 | 2 | 3 | 4 | 5 | 6 | 7 | 8 | 9 |
| Ex. A | 11.00 | 25.00 | 37.00 | 5.00 | 7.50 | 1.10 | 16.50 | 3.00 | 1.80 |
| Ex. B | | 40.00 | 32.00 | 23.00 | 20.00 | 18.00 | 15.00 | 3.00 | 1.00 |
| Ex. C | 19.00 | | 21.00 | 24.00 | 9.00 | 19.00 | 18.60 | 2.20 | 2.80 |
| Ex. D | 10.00 | 20.00 | | 20.00 | 20.00 | 2.90 | 3.90 | 18.00 | 10.00 |
| Ex. E | 14.00 | 24.00 | 34.00 | | 20.00 | 19.00 | 18.00 | 14.00 | 10.00 |
| Ex. F | 5.00 | 20.00 | 20.00 | 20.00 | | 20.00 | 4.00 | 2.50 | 8.00 |
| Ex G | 7.00 | 38.00 | 40.00 | 4.50 | 10.00 | | 18.00 | 17.00 | 2.00 |
| Ex. H | 23.00 | 19.00 | 20.00 | 5.50 | 14.50 | 19.00 | | 3.00 | 2.40 |
| Ex. I | 5.00 | 41.00 | 23.00 | 25.00 | 25.00 | 2.00 | 4.00 | | 2.00 |
| Ex. J | 23.50 | 20.00 | 38.00 | 5.00 | 5.00 | 1.00 | 1.50 | 15.00 | |
| Ex. K | 10.00 | 21.00 | 31.00 | 21.00 | 22.00 | 19.00 | | 10.00 | |
| Ex. L | | 35.00 | 20.00 | 20.00 | 7.00 | 3.00 | | 10.00 | |
| Ex. M | | 19.00 | 20.00 | 25.00 | 10.00 | | | 8.50 | |
| Ex. N | | 40.00 | 20.00 | 30.00 | 20.00 | | | | |

Precursor Exs. A-N are added to cold water (5-12 times the weight of the precursor composition and boiled for 60-90 minutes at room pressure and filtered after boiling. The separated aqueous phases from Exs. A-N are optionally concentrated by evaporation to a paste or power. The decoctions from Exs. A-N or paste or power are useable for administration and for further formulation into disinfectants.

Other embodiments will be apparent to those skilled in the art from consideration of the specification and practice of the present description. It is intended that the specification and examples be considered as exemplary only.

1. A method of treating a patient in need of treatment for coronavirus infection, comprising administering to the patient an effective amount of the composition comprising, a decoction of a precursor composition comprising one or more ingredients chosen from: Lonicera japonica; Forsythia suspensa; Panax ginseng; Schizonepeta tenuifolia; Scrophularia ningpoensis; Prunus armeniaca; honeycomb; Gleditsia sinensis; and Glycyrrhiza uralensis.

2. The method of claim 1, wherein the coronavirus infection is a SARS-CoV infection.

3. The method of claim 1, wherein the coronavirus infection is from a SARS-CoV-2 infection.

4. The method of claim 1, wherein the coronavirus infection is from COVID-19.

5. The method of claim 1, wherein the composition comprises a decoction of a precursor composition comprising Lonicera japonica; Forsythia suspensa; Panax ginseng; Schizonepeta tenuifolia; Scrophularia ningpoensis; Prunus armeniaca; at least one honeycomb chosen from Polistes mandarinus Saussure, P. olivaceus, P. japonicus Saussure, and Parapolybia varia Fabricius; Gleditsia sinensis; and Glycyrrhiza uralensis; or
a decoction of a precursor composition comprising the ingredients of composition #2 to 511 in table 1 (FIG. 8), in which at least one honeycomb is chosen from Polistes mandarinus Saussure, P. olivaceus, P. japonicus Saussure, and Parapolybia varia Fabricius.

6. The method of claim 5, wherein the composition is in the form of a powder or paste made from the decoction whose supernatant aqueous phase is separated from the nonsoluble part of the precursor composition and is at least partially evaporated.

7. The method of claim 6, wherein the supernatant aqueous phase is separated from the nonsoluble part of the precursor composition and is evaporated to dryness.

8. The method of claim 5, wherein the composition further comprises a pharmaceutically or dietary acceptable excipient.

9. The method of claim 8, wherein the pharmaceutically or dietary acceptable excipient is chosen from solvents, binders, lubricants, herbal carriers, oils and salts.

10. The method of claim 5, wherein the composition is in a form suitable for oral administration.

11. The method of claim 10, wherein the form suitable for oral administration is chosen from tablets, pellets, lozenges, granules, capsules, solutions, emulsions, and suspensions.

12. The method of claim 11, wherein the form suitable for oral administration is a dietary supplement.

13. A precursor composition, comprising Lonicera japonica; Forsythia suspensa; Panax ginseng; Schizonepeta tenuifolia; Scrophularia ningpoensis; Prunus armeniaca; honeycomb; Gleditsia sinensis; and Glycyrrhiza uralensis; or
a precursor composition #2 to 511 in table 1 (FIG. 8).

14. A composition in the form of a powder made from the decoction whose supernatant aqueous phase is separated from the nonsoluble part of the precursor composition of claim 13 and is at least partially evaporated to form a powder or paste.

15. A composition of claim 14, wherein the supernatant aqueous phase is separated from the nonsoluble part of the precursor composition and is evaporated to form a powder.

16. The composition of claim 13, wherein the composition further comprises a pharmaceutically or dietary acceptable excipient.

17. The composition of claim 16, wherein the pharmaceutically or dietary acceptable excipient is chosen from solvents, binders, lubricants, herbal carriers, oils and salts.

18. The composition of claim 13, wherein the composition is in a form suitable for oral administration.

19. The composition of claim 13, wherein the precursor composition comprises Lonicera japonica; Forsythia suspensa; Panax ginseng; Schizonepeta tenuifolia; Scrophularia ningpoensis; Prunus armeniaca; at least one honeycomb chosen from Polistes mandarinus Saussure, P. olivaceus, P. japonicus Saussure, and Parapolybia varia Fabricius; Gleditsia sinensis; and Glycyrrhiza uralensis; or
the ingredients of composition #2 to 511 in table 1 (FIG. 8), in which at least one honeycomb is chosen from Polistes mandarinus Saussure, P. olivaceus, P. japonicus Saussure, and Parapolybia varia Fabricius.

20. (canceled)
